(19) United States
(12) Patent Application Publication
(10) Pub. No.: US 2025/0325447 A1
(43) Pub. Date: Oct. 23, 2025

(54) CLOSURE CAP FOR A MEDICAL DEVICE, SAID CAP BEING SUITABLE FOR MAGNETIC GRIP

(71) Applicant: A. Raymond et Cie, Grenoble (FR)

(72) Inventors: Pascal Sircoulomb, Jarrie (FR); Gaëtan Rey, Voiron (FR); Maxime Clavel, Vif (FR); Stéphanie Pellet, Sassenage (FR); Jean-Paul Yonnet, Meylan (FR); Marc De Bortoli, Artiguelouve (FR); Linda Malaquin, Saint Martin D'uriage (FR); Philippe Tenaud, La Combe De Lancey (FR)

(21) Appl. No.: 18/711,480

(22) PCT Filed: Aug. 24, 2022
(86) PCT No.: PCT/EP2022/073601

§ 371 Date: Oct. 7, 2024

(30) Foreign Application Priority Data
Nov. 17, 2021 (FR) 2112141

(51) Int. Cl. A61J 1/14; A61J 1/06; A61L 31/02

(52) U.S. Cl. CPC A61J 1/1412; A61J 1/065; A61L 31/022; A61L 31/026

(57) ABSTRACT

The invention relates to a closure cap (2) for a flask-type medical device, comprising: —an elastomeric stopper intended to be inserted into the neck of the flask after it has been filled with a pharmaceutical or chemical product; —a plastic locking cuff surrounding the stopper and intended to be locked under a collar of the flask when the stopper is inserted into the neck; and —a magnetic material (24) secured to or fully or partially integrated into the stopper or the locking cuff.

### CROSS-REFERENCE TO RELATED APPLICATIONS

This application is a national phase entry under 35 U.S.C. § 371 of International Patent Application PCT/EP2022/073601, filed Aug. 24, 2022, designating the United States of America and published as International Patent Publication WO 2023/088587 Al on May 25, 2023, which claims the benefit under Article 8 of the Patent Cooperation Treaty of French Patent Application Serial No. FR2112141, filed Nov. 17, 2021.

### TECHNICAL FIELD

The present disclosure relates to the field of medical devices and in particular to the field of industrial processes requiring the manipulation of medical device components, up to the point of their packaging for the end user. It relates in particular to a closure cap for a medical device, the cap being suitable for magnetic grip.

### BACKGROUND

A medical device may be composed of a container (for example a flask), intended to be filled with a pharmaceutical or chemical product, and a closure cap, intended to close the container hermetically and securely (that is to say, ensuring tamper-proof locking until the final use).

The flask of a medical device, provided to a laboratory, usually goes through an industrial filling line before being closed and locked by the closure cap. The flasks (respectively the caps) are supplied in batches, in dedicated sterile containers, wherein the flasks (respectively the caps) are held conventionally by plates, as is for example the case in document EP2753550.

The handling of the flasks and caps, in the filling line, is carried out by mechanical gripping (clamping) or more often by suction (suction nozzle) because the latter option is less likely to scratch or damage the parts.

Suction systems, in particular for handling caps, nevertheless have the disadvantage of sucking in potential chemical, or even toxic vapors that are released by the product introduced into the flasks, before their closure; there is therefore a risk that these vapors are then released in an uncontrolled manner at the outlet of the suction systems.

The present disclosure aims to remedy the aforementioned drawback in whole or in part. The present disclosure relates to a closure cap for a flask-type medical device, enabling the hermetic closure of the flask filled with a pharmaceutical or chemical product, and its locking, the closure cap being suitable for magnetic grip, in particular in industrial or chemical product filling lines.

### BRIEF SUMMARY

The present disclosure relates to a closure cap for a flask-type medical device, comprising:
an elastomeric stopper intended to be inserted into a neck of a flask after it has been filled with a pharmaceutical or chemical product,
a plastic locking cuff surrounding the stopper and intended to be locked under a collar of the flask when the stopper is inserted into the neck,
a magnetic material secured to or fully or partially integrated into the stopper or the locking cuff.

According to other advantageous non-limiting features of the present disclosure, taken alone or according to any technically feasible combination:
the magnetic material forms a permanent magnet, in particular a neodymium-iron-boron NdFeB magnet or a ferrite magnet;
the magnetic material is ferromagnetic, selected in particular from iron, cobalt, nickel, or alloys thereof;
the magnetic material is ferrimagnetic, selected in particular from garnet or spinel ferrite;
the locking cuff comprises, at an upper part, a capsule, and the magnetic material is integral with or integrated in whole or in part into the capsule;
the capsule is formed by a plastic material, and a part formed in the magnetic material is glued to a face of the capsule or forcibly inserted into a receptacle provided on a face of the capsule;
the capsule is formed by a plastic material and a part formed in the magnetic material is encapsulated in the plastic material of the capsule;
the capsule is formed of a plastic material filled with particles made of the magnetic material.

The present disclosure also relates to a set of closing caps as above, the caps being arranged in a packaging container comprising at least one tray defining a plurality of receptacles, each closing cap being placed in a receptacle such that the magnetic material is able to interact with a magnetic gripping tool.

The present disclosure finally relates to a medical device comprising:
a closure cap as mentioned above; and
a flask filled with a pharmaceutical or chemical product, hermetically closed by the closure cap.

### BRIEF DESCRIPTION OF THE DRAWINGS

Other features and advantages of the present disclosure will emerge from the following detailed description of the present disclosure with reference to the appended figures, wherein:

FIGS. 1A and 1B show a closure cap according to the present disclosure, and a flask, the assembly forming a medical device, in FIG. 1A, the cap is in the position of temporarily holding on the flask, in FIG. 1B, the cap is in the locked position on the flask;

FIG. 2 shows a plurality of closure caps, according to the present disclosure, packaged in a delivery container and suitable for magnetic gripping.

### DETAILED DESCRIPTION

The present disclosure relates to a closure cap 2 for a flask-type medical device 10.

In the field of pharmaceutical applications, there are standards in terms of the internal diameter of the neck 12 of the flask: 13 mm and 20 mm are examples of this.

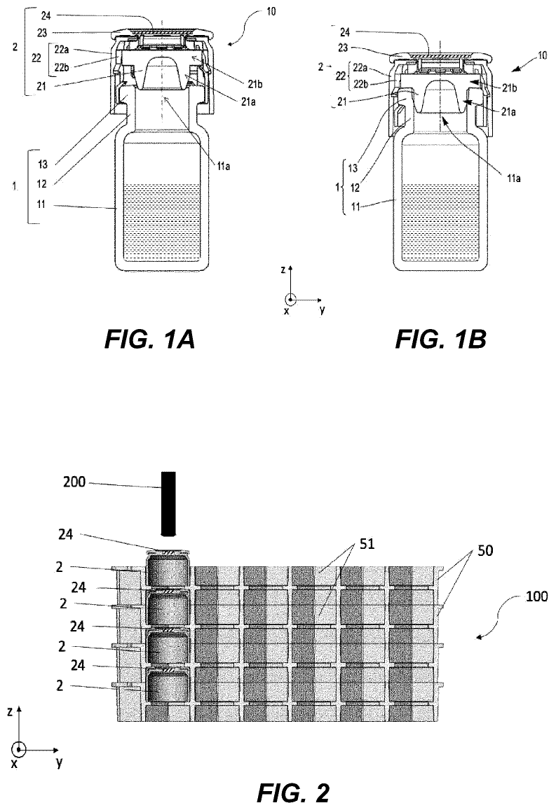

The closure cap 2 comprises, on the one hand, a stopper 21 made of elastomeric material, intended to be inserted into the neck 12 of the flask 1, after the filling thereof with a pharmaceutical or chemical product. On the other hand, it comprises a locking cuff 22 made of plastic material surrounding the stopper 21 and intended to be locked under the collar 13 of the flask 1 when the stopper 21 is inserted into the neck 12. More specifically, as can be seen in FIG. 1B, in the locked position of the closure cap 2, the foot 21a of the stopper 21 is fully inserted into the neck 12, and hermetically seals the opening of the flask 1. The head 21b of the stopper 1 remains pressed on the collar 13. A clip or abutment system, provided on the inner walls of the locking cuff 22, grips under the collar 13, immobilizing the stopper 21 in the opening of the flask 1 and thus securing the closure of the medical device 10.

In FIG. 1A, the closure cap 2 is only placed on the flask 1, in the temporary holding position.

The plastic material constituting the locking cuff 22 may for example be polycarbonate (PC), polypropylene (PP) or polybutylene terephthalate (PBT).

As shown in FIGS. 1A and 1B, the locking cuff 22 of the closure cap 2 may comprise an external body 22a (or ring) and a cage 22b (or wire covering), the latter being configured to engage and lock axially in the external body 22a. In the temporary holding position (FIG. 1A), it is advantageous for flexible tongues, or another holding element forming part of the cage 22b, to block the stopper 21 inside the cage 22b. Other flexible tongues can be arranged in the cage 22b to bear on the collar 13 of the flask 1, when the closure cap 2 is in the temporary holding position; in the final closed position, these other flexible tongues are locked under the collar 13 (FIG. 1B).

The locking cuff 22 may also comprise a capsule 23, at its upper part; in particular, in the example of FIGS. 1A and 1B, the capsule 23 is integral with the external body 22a. It may for example be formed of one of the plastic materials previously mentioned for the locking cuff 22.

This capsule 23 is intended to be irreversibly separated from the closure cap 2, to allow access to the head 21b of the stopper 21: this occurs when the product, contained in the finally-closed medical device 10, is to be taken by the end user. Alternatively, the closure cap 2 can be separable to allow the user to remove it, so as to access the stopper 21 and take the product.

Of course, such a locking cuff 22 is given by way of example, and other configurations could be implemented in order to hold the stopper 21 on the flask 1, while providing a means for accessing the product held in the body 11 of the flask 1, for the end user.

The closure cap 2 further comprises a magnetic material 24, intended to cooperate with a magnetic gripping tool, in particular in industrial filling lines for pharmaceutical or chemical products.

According to a first variant, the magnetic material forms a permanent magnet, in particular a neodymium-iron-boron NdFeB magnet or a ferrite magnet;

According to a second variant, the magnetic material is ferromagnetic and is chosen in particular from iron, cobalt, nickel, or alloys containing them, or ferrimagnetic, and is chosen in particular from garnet ferrites and spinel ferrites.

Considering the disposable (single-use) nature of the closure caps 2, this second variant may prove to be more economical since several soft magnetic materials are easily procured and are inexpensive.

The magnetic material 24 may take the form of a part assembled or integrated with the closure cap 2, or take the form of particles mixed into at least one plastic material making up the closure cap 2.

Advantageously, the magnetic material 24 is integral with the locking cuff 22 while being arranged on or integrated into the closure cap 2.

It should also be noted that it could also be conceivable to place the magnetic material 24 onto or integrate it into the stopper 21; however, due to the direct proximity of the stopper 21 with the pharmaceutical or chemical product contained in the flask 1, it is easier in terms of health standards, to associate the magnetic material with the locking cuff 22.

When the locking cuff 22 comprises a capsule 23, the magnetic material 24 can be arranged on a face of the capsule 23 or integrated therein. The capsule 23 is formed of a plastic material; a part formed in the magnetic material 24 can therefore be rigidly connected to the capsule 23 or integrated onto or into same. According to a first option, the part made of magnetic material 24 is completely or partially overmolded (encapsulated) in the capsule 23, during the molding of the capsule 23. According to a second option, the part is bonded to an upper face or lower face of the capsule 23 (in the latter case, to be between the capsule 23 and the external body 22a of the locking cuff 22), after molding of the capsule 23. Finally, in a third option, the part made of magnetic material 24 is forcibly fitted into a receptacle, produced during molding, at the upper face or the lower face of the capsule 23.

According to any one of these three options, the part made of magnetic material 24 can take any shape, provided that it is adapted to the combination of the part on or in the capsule 23. Mention may in particular be made of disc, ring or other polygonal shapes in the plane (x, y) that is the main plane of the capsule 23.

Alternatively, the capsule 23 may be formed from a plastic material filled with particles made of the magnetic material 24.

When the locking cuff 22 does not comprise a capsule 23 (the external body 22a is then closed in its upper part), the magnetic material 24 is arranged on or integrated into the upper part of the external body 22a, at the center or at the periphery, in any shape, for example a disc, ring or other polygonal shape in the plane (x, y).

The external body 22a consists of a plastic material; a part formed from the magnetic material 24 can be integral with the external body 22a or wholly or partially integrated therein. The aforementioned first, second and third options can be applied, namely:
the part made of magnetic material 24 is completely or partially overmolded in the external body 22a, during the molding thereof; or
the part is bonded to or under the upper part of the external body 22a (in the latter case, to be between the external body 22a and the cage 22b), after the molding of said the external body 22a; or
the part made of magnetic material 24 is force-fitted into a receptacle, produced during molding, on or under the upper part of the external body 22a.

Alternatively, the capsule 23 may be formed from a plastic material filled with particles made of the magnetic material 24.

It should be noted that the magnetic material 24 could also be integral with or integrated in all or part in the cage 22b, according to the same options as those described above.

The volume and magnetic properties of the magnetic material 24 are sized so that the magnetic attraction between the magnetic material 24 and a magnetic gripping tool is sufficient to lift and manipulate the closure cap 2 in an industrial line, for example during a process for filling and closing flasks 1 for medical devices 10.

The closure cap 2 according to the present disclosure is compatible with a magnetic gripping tool 200, as shown in FIG. 2. Such a tool can take various shapes, but comprises at least one end provided with a magnet or an electromagnet or formed of soft ferromagnetic material.

In the case of an electromagnet, the latter is activated at the time of the extraction of the closure cap 2 from its packaging plate 50 until it is positioned on the flask 1. It is then deactivated, once the closure cap 2 is placed on the flask 1 (FIG. 1A) or, once the closure cap 2 is permanently locked to the flask 1 (FIG. 1B).

In the case of a single magnet placed at the end of a shaft, the shaft must be strong enough to allow the extraction and conveying of the closure cap 2 without risk of falling, until it has been positioned on the flask 1, and small enough so that once the closure cap 2 has been locked onto the flask 1, the magnet of the tool releases it, without lifting the medical device 10 and without pulling off the removable capsule 23 when it is present.

According to the present disclosure, the closure caps 2 can be packaged in the form of a set 100 of closure caps 2. For this, the closure caps 2 are arranged in a packaging container (not shown), which comprises at least one packing plate 50 defining a plurality of receptacles 51 (FIG. 2). Each closure cap 2 is placed in a receptacle 51 so that the magnetic material 24 is able to cooperate with the magnetic gripping tool 200.

When the flask 1 is filled with the pharmaceutical product, the magnetic gripping tool 200 takes a closure cap 2 on the packing plate 50, and places it on the flask 1, in the temporary holding position shown in FIG. 1A. The magnetic gripping tool 200 can then be configured to release the closure cap 2, which will be pushed into the locked position on the flask 1 by applying a bearing force, for example by a press subsequently intervening in the filling line. Alternatively, the magnetic gripping tool 200 can also be configured to apply the bearing force and lock the closure cap 2 on the flask 1, before releasing the magnetic plug.

The closure cap 2 according to the present disclosure prevents potential chemical vapors from being sucked in, while allowing reliable and practical gripping in an industrial line.

The present disclosure further relates to the medical device 10 shown in FIG. 1B, which comprises a closure cap 2 as described above and a flask 1 filled with a pharmaceutical or chemical product and hermetically closed by the closure cap 2.

Optionally, the sizing (volume and magnetic properties) of the magnetic material 24 included in the closure cap 2 can be defined so as to allow the conveying and the gripping of the medical device 10, for example, to place said the medical device 10, after closing the flask 1, into a collective repackaging container or to extract it from such a container at the end of the final use.

Naturally, the present disclosure is not limited to the embodiments and examples that have been described, and it is possible to add alternative embodiments thereto without departing from the scope of the invention as defined by the claims.

1. A closure cap (2) for a flask-type medical device (10), comprising:
an elastomeric stopper (21) intended to be inserted into a neck (12) of a flask (1) after it has been filled with a pharmaceutical or chemical product,
a plastic locking cuff (22) surrounding the stopper (21) and intended to lock under a collar (13) of the flask (1) when the stopper (21) is inserted into the neck (12), the locking cuff (22) comprising, at an upper part, a capsule (23),
a magnetic material (24) fully or partially integrated into the capsule (23);
and wherein:
the capsule (23) is formed by a plastic material and a part formed from the magnetic material (24) is fully or partially encapsulated in the plastic material of said capsule (23), or
the capsule (23) is formed of a plastic material filled with particles made of the magnetic material (24).

2. The closing cap (2) according to the preceding claim, wherein the magnetic material (24) forms a permanent magnet, in particular a neodymium-iron-boron NdFeB magnet or a ferrite magnet.

3. The closing cap (2) according to claim 1, wherein the magnetic material (24) is ferromagnetic, selected in particular from iron, cobalt, nickel, or alloys containing them.

4. The closing cap (2) according to claim 1, wherein the magnetic material (24) is ferrimagnetic, selected in particular from among garnet or spinel ferrites.

5. A set (100) of closing caps (10) according to any of the preceding claims, said caps (10) being arranged in a packaging container comprising at least one tray (50) defining a plurality of receptacles (51), each closing cap (2) being placed in a receptacle (51) such that the magnetic material (24) is able to interact with a magnetic gripping tool (200).

6. A medical device (10) comprising:
a closing cap (2) according to one of claims 1 to 4, and
a flask (1) filled with a pharmaceutical or chemical product, hermetically closed by said closure cap (2).
